(19) United States
(12) Patent Application Publication
(10) Pub. No.: US 2008/0167696 A1
(43) Pub. Date: Jul. 10, 2008

(54) STIMULUS WAVEFORMS FOR BAROREFLEX ACTIVATION

(75) Inventors: Adam W. Cates, Minneapolis, MN (US); Martin A. Rossing, Coon Rapids, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Minneapolis, MN (US)

(21) Appl. No.: 11/963,357

(22) Filed: Dec. 21, 2007

Related U.S. Application Data
(60) Provisional application No. 60/882,478, filed on Dec. 28, 2006.

(51) Int. Cl. A61N 1/18

(52) U.S. Cl. 607/44

(57) ABSTRACT

A method and apparatus for stimulation of a baroreflex system of a patient is provided. A method comprises establishing a therapy regimen including at least one pulse which includes at least two phases. Each phase has a polarity which is different than that of the other phase. The baroreflex system of the patient is activated with at least one baroreflex activation device which is responsive to the therapy regimen.

### CROSS-REFERENCES TO RELATED APPLICATIONS

The present application claims the benefit of provisional U.S. Application No. 60/882,478 (Attorney Docket No. 021433-000300US), filed Dec. 28, 2006, the full disclosure of which is incorporated herein by reference.

This application is related to, but does not claim the benefit of the following U.S. patents and applications, all of which are is fully incorporated herein by reference in their entirety: U.S. Pat. Nos. 6,522,926; 6,616,624; 6,985,774; 7,158,832; 6,850,801; PCT Patent Application No. PCT/US01/30249, filed Sep. 27, 2001 (Attorney Docket No. 21433-000140PC); U.S. patent application Ser. Nos. 10/284,063 (Attorney Docket No. 21433-000150US), filed Oct. 29, 2002; 10/453,678 (Attorney Docket No. 21433-000210US), filed Jun. 2, 2003; 10/402,911 (Attorney Docket No. 21433-000410US), filed Mar. 27, 2003; 10/402,393 (Attorney Docket No. 21433-000420US), filed Mar. 27, 2003; 10/818,738 (Attorney Docket No. 21433-000160US), filed Apr. 5, 2004; and 60/584,730 (Attorney Docket No. 21433-001200US), filed Jun. 30, 2004; 11/168,231 (Attorney Docket No. 21433-001210US), filed Jun. 27, 2005; and 10/958,694 (Attorney Docket No. 21433-001600US), filed Oct. 4, 2004.

### BACKGROUND OF THE INVENTION

1. Field of Invention

The present invention relates generally to medical devices and methods of use for the treatment and/or management of cardiovascular, neurological, and renal disorders, and more specifically to devices and methods for controlling the baroreflex system for the treatment and/or management of cardiovascular, neurological, and renal disorders and their underlying causes and conditions.

Hypertension, or high blood pressure, is a major cardiovascular disorder that is estimated to affect 65 million people in the United Sates alone, and is a leading cause of heart failure and stroke. It is listed as a primary or contributing cause of death in over 200,000 patients per year in the United States alone. Hypertension occurs in part when the body's smaller blood vessels (arterioles) constrict, causing an increase in blood pressure. Because the blood vessels constrict, the heart must work harder to maintain blood flow at the higher pressures. Sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke.

Sustained high blood pressure may eventually result in an enlarged and damaged heart (hypertrophy), which may lead to heart failure. Heart failure is the final common expression of a variety of cardiovascular disorders, including ischemic heart disease. It is characterized by an inability of the heart to pump enough blood to meet the body's needs and results in fatigue, reduced exercise capacity and poor survival. It is estimated that approximately 5,000,000 people in the United States suffer from heart failure, directly leading to 39,000 deaths per year and contributing to another 225,000 deaths per year.

A number of drug treatments have been proposed for the management of hypertension, heart failure, and other cardiovascular disorders. These include vasodilators to reduce the blood pressure and ease the workload of the heart, diuretics to reduce fluid overload, inhibitors and blocking agents of the body's neurohormonal responses, and other medicaments. Various surgical procedures have also been proposed for these maladies. For example, heart transplantation has been proposed for patients who suffer from severe, refractory heart failure. Alternatively, an implantable medical device such as a ventricular assist device (VAD) may be implanted in the chest to increase the pumping action of the heart. Alternatively, an intra-aortic balloon pump (IABP) may be used for maintaining heart function for short periods of time, but typically no longer than one month.

Although each of these approaches is beneficial in some ways, each of the therapies has its own disadvantages. For example, drug therapy is often incompletely effective. Drugs often have unwanted side effects and may need to be given in complex regimens. These and other factors contribute to poor patient compliance with medical therapy. Drug therapy may also be expensive, adding to the health care costs associated with these disorders.

2. Brief Description of the Background Art

It has been known for decades that the wall of the carotid sinus, a structure at the bifurcation of the common carotid arteries, contains stretch receptors (baroreceptors) that are sensitive to the blood pressure. These receptors send signals via the carotid sinus nerve to the brain, which in turn regulates the cardiovascular system to maintain normal blood pressure (the baroreflex), in part through modulation of the sympathetic and/or parasympathetic, collectively the autonomic, nervous system. Electrical stimulation of the carotid sinus nerve (baropacing) has previously been proposed to reduce blood pressure and the workload of the heart in the treatment of high blood pressure and angina.

Rau et al. (2001) Biological Psychology 57:179-201 describes animal and human experiments involving baroreceptor stimulation. U.S. Pat. Nos. 6,073,048 and 6,178,349, each having a common inventor with the present application, describe the stimulation of nerves to regulate the heart, vasculature, and other body systems. U.S. Pat. No. 6,522,926, assigned to the assignee of the present application, describes a number of systems and methods intended to activate baroreceptors in the carotid sinus and elsewhere in order to induce the baroreflex system. Numerous specific approaches are described, including the use of coil electrodes placed over the exterior of the carotid sinus near the carotid bifurcation. Nerve stimulation for other purposes is described in, for example, U.S. Pat. Nos. 6,292,695 B1 and 5,700,282. Publications which describe the existence of baroreceptors and/or related receptors in the venous vasculature and atria include Goldberger et al. (1999) J. Neuro. Meth. 91:109-114; Kostreva and Pontus (1993) Am. J. Physiol. 265:G15-G20; Coleridge et al. (1973) Circ. Res. 23:87-97; Mifflin and Kunze (1982) Circ. Res. 51:241-249; and Schaurte et al. (2000) J. Cardiovasc Electrophysiol. 11:64-69. U.S. Pat. No. 5,203,326 describes an anti-arrhythmia pacemaker. PCT patent application publication number WO 99/51286 describes a system for regulating blood flow to a portion of the vasculature to treat heart disease. The full texts and disclosures of all the references listed above are hereby incorporated fully by reference in their entirety.

Cardiac resynchronization therapy (CRT) devices are known. Examples of CRT devices and methods are described in U.S. Pat. Nos. 6,768,923; 6,766,189; 6,748,272; 6,704,598; 6,701,186; and 6,666,826; the full disclosures of which are hereby incorporated by reference in their entirety.

An example of an implantable blood pressure measurement device that may be disposed about a blood vessel is disclosed in U.S. Pat. No. 6,106,477 to Miesel et al. An example of a subcutaneous ECG monitor is available from Medtronic under the trade name REVEAL ILR and is disclosed in PCT Publication No. WO 98/02209. Other examples are disclosed in U.S. Pat. Nos. 5,987,352 and 5,331,966. Examples of devices and methods for measuring absolute blood pressure utilizing an ambient pressure reference are disclosed in U.S. Pat. No. 5,810,735 to Halperin et al., U.S. Pat. No. 5,904,708 to Goedeke, and PCT Publication No. WO 00/16686 to Brockway et al. The full texts and disclosures of all the references listed above are hereby incorporated fully by reference in their entirety.

### SUMMARY OF THE INVENTION

To address the problems of hypertension, heart failure, other cardiovascular disorders, nervous system and renal disorders, the present invention provides methods, and devices (i.e., baroreflex activation device) for practicing the same, by which at least one baroreflex system within a patient's body is activated to achieve effects that include reducing excessive blood pressure, autonomic nervous system activity, and neurohormonal activation. Such activation systems suggest to the brain an increase in blood pressure and the brain in turn regulates (e.g., decreases) the level of sympathetic nervous system and neurohormonal activation, and increases parasypathetic nervous system activation, thus reducing blood pressure and having a beneficial effect on the cardiovascular system and other body systems.

The methods and devices according to the present invention may be used to activate baroreceptors, mechanoreceptors, pressoreceptors, or any other venous heart, or cardiopulmonary receptors which affect the blood pressure, nervous system activity, and neurohormonal activity in a manner analogous to baroreceptors in the arterial vasculation. For convenience, all such venous receptors (and/or nerves carrying signals from such receptors) will be referred to collectively herein as “baroreceptors.”

The therapy regimen for baroreflex activation stimulus is governed by a control system which is selected to promote long term efficacy. It is possible that the uninterrupted or otherwise unchanging activation of baroreceptors and/or nerve fibers that carry signals from the baroreceptor to the brain may result in the baroreceptors and/or the baroreflex system becoming less responsive over time, thereby diminishing the long term effectiveness of the therapy. Therefore, the stimulus regimen maybe selected to activate, deactivate, or otherwise modulate a baroreflex activation device in such a way that therapeutic efficacy is maintained for months, preferably for years. In some embodiments, for example, applying the baroreflex activation stimulus comprises transmitting energy from at least one energy transmitting device stimulating an area approximating one or more carotid arteries. The area may be a carotid sinus.

In an embodiment, the present invention provides a method by which baroreceptors and/or nerve fibers that carry signals from the baroreceptors to the brain may be activated by establishing a therapy regimen including at least one multiphasic pulse. In an embodiment the at least one multiphasic pulse includes at least one biphasic pulse. For discussion purposes, biphasic pulse will be used herein although it should be appreciated that a given pulse may include more than two phases. It should be appreciated that the various phases of the regimen therapy (both inter-pulse and intra-pulse) may have similar or different waveforms as for example different amplitudes, widths, size, and shapes (e.g., square wave or ramp wave, symmetrical or asymmetrical). In an embodiment, the therapy regimen includes applying a plurality of biphasic pulses. Each phase of each biphasic pulse has a polarity which is different than that of the other phase within the same pulse. The baroreflex system of the patient is activated with at least one baroreflex activation device which is responsive to the therapy regimen. In an embodiment, the baroreflex activation device includes an electrode assembly having at least one electrode. In an embodiment, the electrode assembly includes a plurality of electrodes. In an embodiment, the electrode assembly includes at least one set of electrodes where the anode and the cathode are switching during at least one pulse (i.e., at least one electrode switches between behaving as a cathode and an anode). In an embodiment, the electrode assembly includes at least one set of electrodes with a tripolar or pseudotripolar configuration. In an embodiment, the tripolar electrode set includes a central electrode and two outer electrodes, as further described below. However, it should be appreciated that the methods and devices of the present invention may be used with any number of electrodes and configurations, as for example a bipolar electrode, or a monopolar electrode (e.g., an electrode set including an active electrode and a dispersive electrode). For further details of exemplary electrodes useful in the practice of the present invention, reference may be made to U.S. patent application Ser. Nos. 10/402,911 (Attorney Docket No. 21433-000410US), filed Mar. 27, 2003; 10/402,393 (Attorney Docket No. 21433-000420US), filed Mar. 27, 2003; and 10/958,694 (Attorney Docket No. 21433-001600US), filed Oct. 4, 2004; the full disclosures of all of which were previously incorporated by reference in their entirety.

Generally, when electrical stimuli are delivered to the tissue, the tissue beneath each electrode is polarized, with the area of excited tissue beneath the cathodic electrode being larger than that beneath the anodic electrode. Without intending to limit the scope of the present invention, it was found by the present inventors that by reversing the polarity during the course of a given pulse, the tissue around each electrode is directly depolarized. It was further discovered, that this depolarization extends the region of tissue affected by the subsequent stimulation without increasing amplitude or width of the pulse (in contrast for example in a single polarity method). Therefore, employing a biphasic pulse provides a better response for a given energy delivered. Additionally, the use of a biphasic waveform minimizes local hyperpolarization of tissue which otherwise may result from the use of monophasic waveforms which can limit the excitability of the tissue for a subsequent pulse. The second phase of a biphasic waveform, thus, may reduce the hyperpolarization, preparing the excitable tissue for the next pulse.

In an embodiment, each phase is delivered for a predefined duration of time (i.e., predefined phase width). Each phase of the each pulse is separated by an interphase delay of predefined time period (i.e., interphase delay width). The phase width of each pulse may be similar or different from the phase width of the other phase of the same pulse. For example, the first phase of a given pulse may have an equal, shorter, or greater phase width (time duration) than the second phase of the same pulse. Similarly, the phase width may be similar, less, or more than the interphase delay between two successive phases of the same pulse. The phase widths and the interphase delays of different pulses may be similar or different from one another. In some embodiments, the interphase delay between two phases of a given pulse may be equal, shorter, or greater than the time duration between that pulse and another pulse immediately preceding or following that given pulse. In an embodiment, the therapy regimen includes a series of biphasic pulses with the interphase delay between two successively delivered phases being shorter than a time interval between the two adjacently delivered (i.e., two pulses delivered immediately next to each other) biphasic pulses. Alternatively, the interphase delay between two successively delivered phases may be greater than or equal to the time interval between the two adjacently delivered biphasic pulses. In an embodiment, the width of the first phase is effectively shorter than that of the second phase to equilibrate the charge delivered to the baroreflex system in each phase.

In an embodiment, the magnitude of the each phase width and/or the interphase delay, independently, may range from about 30 to about 3000 micro seconds (“μs”), from about 100 to about 1000 μs, from about 200 to about 2000 μs, from about 500 to about 3000 μs, from about 30 to about 500 μs. In an embodiment, the phase width and/or the interphase delay, is about 100 μs. In an embodiment, the biphasic pulse comprises the output of a single discharging capacitor and the polarity is switched midway during the delivery of the pulse such that the first phase and the second phase have equal widths. In an embodiment, the biphasic pulse comprises the output of a constant current source. In an embodiment, the interphase delay is about 100 μs.

In an embodiment, during the therapy regimen, the direction of current flowing through a target baroreflex system alternates between phases of at least one pulse. By way of example, during at least one phase of at least one pulse, current is delivered in one direction through the target baroreflex system thereby producing a positive phase while during another phase of the same pulse, current flows through the target baroreflex system in a direction opposite that of the one direction. Thus, for at least one pulse during the therapy regimen, the polarity of the at least one electrode switches from behaving as a cathode to an anode and/or vice versa.

In an embodiment, the positive phase is the first phase of the biphasic pulse while the negative phase is the second phase of the same biphasic pulse. In an alternate embodiment, the negative phase is the first phase of the biphasic pulse while the positive phase is the second phase of the same biphasic pulse. In an embodiment, each successive pulse always starts with the same polarity. In an embodiment, the polarity of the first phase of the plurality of the biphasic pulses alternates between positive and negative. In an embodiment, biphasic and monophasic stimulation are each provided periodically.

In an embodiment, the electrode assembly delivering the pulses to the target baroreflex system includes at least one set of electrodes having tripolar configuration. In an embodiment, the tripolar electrode set includes a central electrode flanked by two outer electrodes. In an embodiment, at nominal polarity, the central electrode has a different charge than the two outer electrodes. In an embodiment, at nominal polarity the central electrode behaves as a cathode and the outer two electrodes behave as anodes. In some embodiments, the polarity of each electrode is changed at least once during at lease one biphasic pulse. In an embodiment, each given electrode starts the next pulse with a polarity which is the same as that of a previous pulse for that given electrode. Alternatively, the polarity of each given electrode between pulses alternates from one polarity to another opposite polarity.

In an embodiment, a device for activating a baroreflex system of a patient is provided which includes an electrode assembly having a plurality of electrodes configured to be responsive to a therapy regimen applying at least one biphasic pulse to the electrode assembly with each phase imparting to a given electrode a polarity which is opposite that imparted by another phase within the same pulse.

It should be appreciated that methods and devices according to the present invention may be used alone or in combination with other therapy methods and devices to achieve separate, complementary, or synergistic effects. Examples of such other methods and devices include Cardiac resynchronization therapy (CRT), Cardiac Rhythm Management (CRM), anti-arrhythmia treatment as for example applied to the heart via a cardiovertor/defibrillator; drug delivery devices and systems; as well as diagnostic and/or monitoring modalities. The above devices and/or systems, may be separate or integrated into a combination device in which the component therapies perform independently or in concert.

### BRIEF DESCRIPTION OF THE DRAWINGS

FIG. 1 is a schematic illustration of the chest and head regions of a human body showing the major arteries and veins and associated anatomy;

FIG. 2A is a cross-sectional schematic illustration of the carotid sinus and baroreceptors within the vascular wall;

FIG. 2B is a schematic illustration of baroreceptors within a vascular wall, and a schematic flow chart of the baroreflex system;

FIG. 3 is a schematic illustration of a baroreflex activation system applied to a human subject according to an embodiment of the present invention;

FIG. 4A is an exemplary circuit diagram and the corresponding wave form, employing features of the present invention;

FIGS. 4-B-F are other exemplary wave form diagrams employing features of the present invention.

FIGS. 5 and 6 are schematics illustration of an exemplary electrode assembly usable in the practice of the present invention.

FIG. 7 is a more detailed illustration of electrode coils which are present in an elongate lead of the electrode assembly of FIG. 5.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention. The drawings illustrate the specific embodiment where one or more baroreflex activation devices are positioned near baroreceptors. However, as can be appreciated, the invention is applicable to baroreflex activation devices that are positioned near nerve fibers that carry signals from the baroreceptor to the brain.

Anatomical Overview

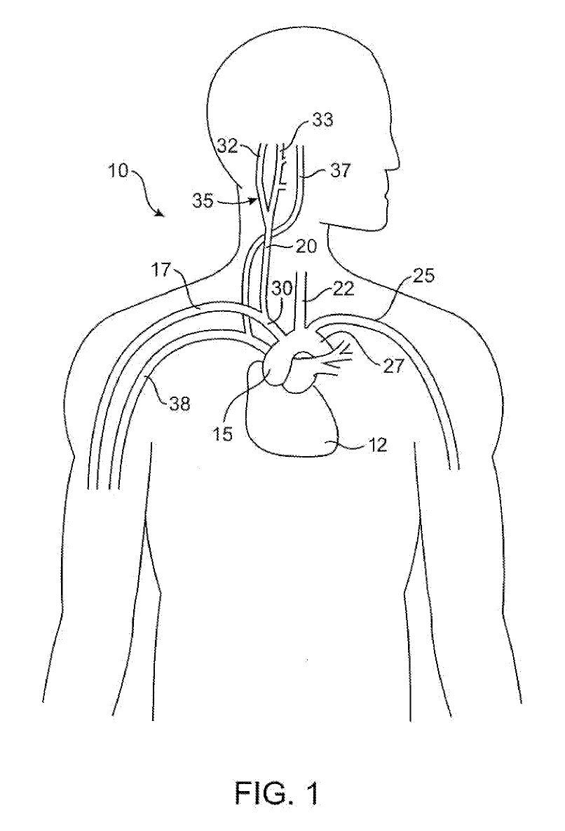

Referring to FIG. 1, chest and head regions of a human body 10 including some of the major arteries and veins of the cardiovascular system are schematically shown. The left ventricle of a heart 12 pumps oxygenated blood up into the aortic arch 15. The right subclavian artery 17, the right common carotid artery 20, the left common carotid artery 22, and the left subclavian artery 25 branch off the aortic arch 15 proximal of the descending thoracic aorta 27. Although relatively short, a distinct vascular segment referred to as the brachiocephalic artery 30 connects the right subclavian artery 17 and the right common carotid artery 20 to the aortic arch 15. The right carotid artery 20 bifurcates into the right external carotid artery 32 and the right internal carotid artery 33 at the right carotid sinus 35. Although not shown for purposes of clarity only, the left carotid artery 22 similarly bifurcates into the left external carotid artery and the left internal carotid artery at the left carotid sinus.

From the aortic arch 15, oxygenated blood flows into the carotid arteries 20/22 and the subclavian arteries 17/25. From the carotid arteries 20/22, oxygenated blood circulates through the head and cerebral vasculature and oxygen-depleted blood returns to the heart 12 by way of the jugular veins, of which only the right internal jugular vein 37 is shown for sake of clarity. From the subclavian arteries 17/25, oxygenated blood circulates through the upper peripheral vasculature and oxygen depleted blood returns to the heart by way of the subclavian veins, of which only the right subclavian vein 38 is shown, also for sake of clarity. The heart 12 pumps the oxygen depleted blood through the pulmonary system where it is re-oxygenated. The re-oxygenated blood returns to the heart 12 which pumps the re-oxygenated blood into the aortic arch as described above, and the cycle repeats.

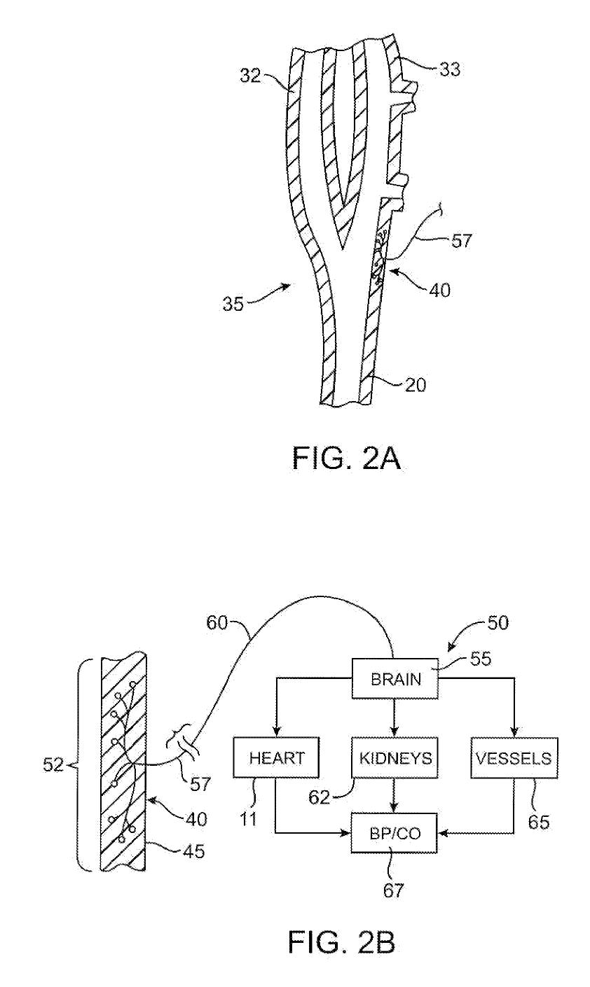

FIG. 2A is a cross-sectional schematic illustration of the right carotid sinus 35 showing the presence of baroreceptors 40 within the vascular wall of the right common carotid artery 20 near the right carotid sinus 35. Baroreceptors are also present, for example, within the arterial walls of the aortic arch 15, the left common carotid artery 22 (near the left carotid sinus), subclavian arteries 17/25, and brachiocephalic artery 30. Baroreceptors 40 are a type of stretch receptor used by the body to sense blood pressure, and exist in both arterial and venous structures. An increase in blood pressure causes the vascular wall to stretch, and a decrease in blood pressure causes the vascular wall to return to its original size. Such a cycle is repeated with each beat of the heart. Because baroreceptors 40 are located within the vascular wall, they are able to sense deformation of the adjacent tissue, which is indicative of a change in blood pressure. As used herein, the term “baroreceptors” is used to refer to baroreceptors in arterial vasculation, as well as mechanoreceptors, pressoreceptors, or any other venous heart, or cardiopulmonary receptors which affect the blood pressure, nervous system activity, and neurohormonal activity in a manner analogous to baroreceptors in the arterial vasculation. For convenience, all such venous receptors (and/or nerves carrying signals from such receptors) whether, in arteries or veins, will be referred to collectively herein as “baroreceptors.” Thus for discussion purposes, it will be assumed that baroreceptors 40 are connected to the brain 55 via the nervous system 60.

FIG. 2B is a schematic illustration of baroreceptors 40 within a generic vascular wall 45 and showing the interaction with the baroreflex system, denoted schematically as 50. The baroreceptors 40 located in the right carotid sinus 35, the left carotid sinus, and the aortic arch 15 play the most significant role in sensing blood pressure that affects baroreflex system 50, which is now described in more detail. Specifically, baroreceptors 40 are profusely distributed within the vascular walls 45 of the major arteries discussed previously, and generally form an arbor 52. Baroreceptor arbor 52 comprises a plurality of baroreceptors 40, each of which transmits baroreceptor signals to the brain 55 via a nerve 57. Baroreceptors 40 are so profusely distributed and arborized within the vascular wall 45 that discrete baroreceptor arbors 52 are not readily discernable. To this end, those skilled in the art will appreciate that baroreceptors 40 shown in FIG. 2B are primarily schematic for purposes of illustration and discussion.

Baroreceptor signals are used to activate a number of body systems which collectively may be referred to as baroreflex system 50. Baroreceptors 40 are connected to the brain 55 via the nervous system 60. Thus, the brain 55 is able to detect changes in blood pressure, which is indicative of cardiac output. If cardiac output is insufficient to meet demand (i.e., the heart 12 is unable to pump sufficient blood), baroreflex system 50 activates a number of body systems, including the heart 12, kidneys 62, vessels 65, and other organs/tissues. Such activation of baroreflex system 50 generally corresponds to an increase in neurohormonal activity. Specifically, baroreflex system 50 initiates a neurohormonal sequence that signals the heart 12 to increase heart rate and increase contraction force in order to increase cardiac output, signals the kidneys 62 to increase blood volume by retaining sodium and water, and signals the vessels 65 to constrict to elevate blood pressure. The cardiac, renal and vascular responses increase blood pressure and cardiac output (denoted schematically at 67), and thus increase the workload of the heart 12. In a patient with heart failure, this further accelerates myocardial damage and exacerbates the heart failure state.

System Overview

To address the problems of hypertension, heart failure, other cardiovascular disorders, nervous system and renal disorders, the present invention provides methods by which baroreflex system 50 is activated to reduce excessive blood pressure, autonomic nervous system activity, and neurohormonal activation. In particular, the present invention provides a method by which baroreceptors 40 and/or nerve fibers that carry signals from the baroreceptors to the brain may be activated in a biphasic mode, described in detail below. Such activation systems signal to the brain 55 the increase in blood pressure and the brain in turn regulates (e.g., decreases) the level of sympathetic nervous system and neurohormonal activation, and increases parasypathetic nervous system activation, thus reducing blood pressure and having a beneficial effect on the cardiovascular system and other body systems.

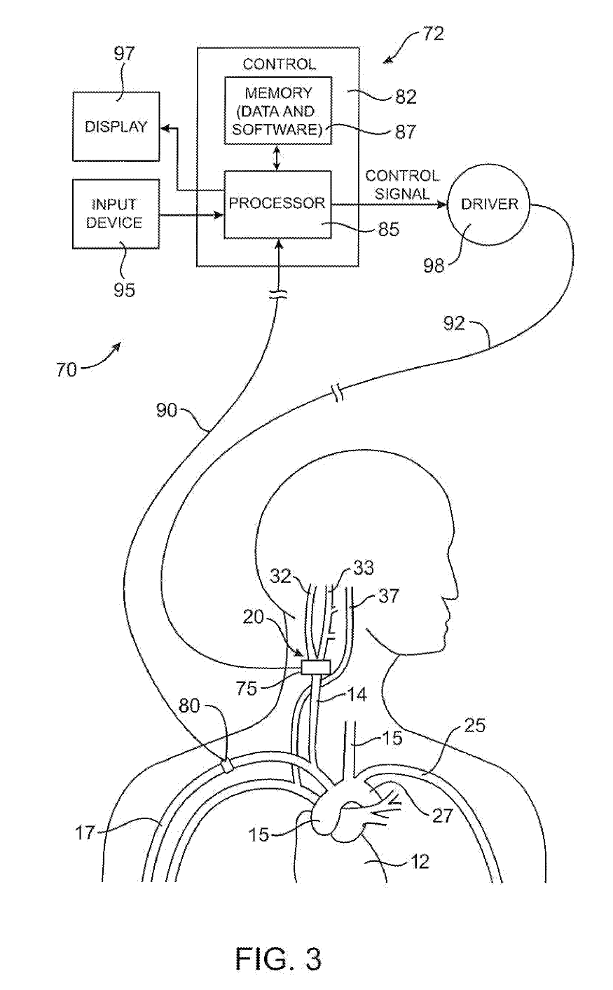

FIG. 3 is a schematic illustration of a baroreflex activation system 70 applied to a human subject according to an embodiment of the present invention. The human subject may be the person shown in FIG. 1, and corresponding reference numbers are used. In brief, baroreflex activation system 70 includes a control system 72, a baroreflex activation device 75, and an optional sensor 80, which generally operate in the following manner. Sensor 80 optionally senses and/or monitors a parameter (e.g., cardiovascular function) indicative of the need to modify the baroreflex system and generates a signal indicative of the parameter. In some embodiments (not shown), sensor 80 may be incorporated into the structure of baroreflex activation device 75.

Control system 72 generates a control signal that activates, deactivates, or otherwise modulates baroreflex activation device 75. Typically, activation of baroreflex activation device 75 results in activation of baroreceptors 40 and/or nerve fibers that carry signals from the baroreceptor to the brain. Alternatively, deactivation or modulation of baroreflex activation device 75 may cause or modify activation of baroreceptors 40 and/or nerve fibers (such as carotid sinus nerve fibers) that carry signals from the baroreceptor to the brain. Control system 72 may generate the control signal according to a predetermined schedule or in response to human action.

For embodiments using optional sensor 80, the control system can generate the control signal as a function of the received sensor signal. This could be independent of a predetermined schedule, or as an adjunct to the schedule. For example, if sensor 80 were to detect a parameter indicative of the need to modify the baroreflex system activity (e.g., excessive blood pressure), control system 72 would cause the control signal to modulate (e.g., activate and/or increase) baroreflex activation device 75, thereby inducing a signal from baroreceptor 40 and/or nerve fibers near the baroreceptor to the brain that is perceived by the brain 55 to be apparent excessive blood pressure. When sensor 80 detects a parameter indicative of normal body function (e.g., normal blood pressure), control system 72 would cause the control signal to modulate (e.g., deactivate and/or decrease) baroreflex activation device 75. The sensor and control system may also be used to control timing of the delivery of the therapy, for example being R-wave triggered, and/or they may also dictate the timing or intensity of the therapy relative to a respiratory cycle. The sensor may also determine the sidedness of the therapy (for example in the presence of atrial fibrillation versus Normal Sinus Rhythm).

By way of example, control system 72 includes a control block 82 comprising a processor 85 and a memory 87. Control system 72 is connected to sensor 80 by way of a sensor cable 90. Control system 72 is also connected to baroreflex activation device 75 by way of a control cable 92. Thus, control system 72 receives a sensor signal from sensor 80 by way of sensor cable 90, and transmits a control signal to baroreflex activation device 75 by way of control cable 92. Control system 72 is also typically provided with an input device 95 and an output device or display 97. Some embodiments generate a control signal that includes trains of short pulses. While the embodiments are not limited to any particular circuitry for generating such pulses, it is noted that a suitable form of pulse generator could include one or more switches, such as field-effect transistor (FET) switches, controlled by processor 85 to connect one or more programmable voltage power supplies to the output.

System components 72/75/80 may be directly linked via cables 90/92 or by indirect means such as RF signal transceivers, ultrasonic transceivers, or galvanic couplings. Examples of such indirect interconnection devices are disclosed in U.S. Pat. No. 4,987,897 to Funke and U.S. Pat. No. 5,113,859 to Funke, the entire disclosures of which are incorporated herein by reference. In some instances, control system 72 includes a driver 98 to provide the desired power mode for baroreflex activation device 75. For example, the driver 98 may comprise a power amplifier or the like and cable 92 may comprise electrical lead(s). In other instances, driver 98 may not be necessary, particularly if processor 85 generates a sufficiently strong electrical signal for low level electrical actuation of baroreflex activation device 75. The electrode structure may receive electrical signals directly from the driver 98 of the control system 72 by way of electrical lead 92, or indirectly by utilizing an inductor (not shown) as described in copending commonly assigned application Ser. No. 10/402,393 (Attorney Docket No. 21433-000420); as well as various electrode designs as described in copending commonly assigned application Ser. No. 10/402,911 (Attorney Docket No. 21433-000410); both filed on Mar. 27, 2003, the full disclosures of which are incorporated herein by reference.

Representative Baroreflex Activation Devices

Baroreflex activation device 75 may directly activate one or more baroreceptors 40 by changing the electrical potential across baroreceptors 40. It is also possible that changing the electrical potential might activate nerve fibers, or might indirectly change the thermal or chemical potential across the tissue surrounding baroreceptors 40 and/or otherwise may cause the surrounding tissue to stretch or otherwise deform, thus mechanically activating baroreceptors 40 and/or nerve fibers that carry signals from the baroreceptor to the brain. Thus, baroreflex activation device 75 activates baroreceptors 40 and/or nerve fibers that carry signals from the baroreceptor to the brain electrically, optionally in combination with mechanical, thermal, chemical, biological or other co-activation. Thus, when control system 72 generates a control signal to modulate (e.g., activate) baroreflex activation device 75, this induces a signal from baroreceptor 40 and/or nerve fibers that carry signals from the baroreceptor to the brain that presumably are perceived by the brain 55 to be apparent excessive blood pressure, and the baroreflex system operates to lower the blood pressure. However, it is generally contemplated that the control signal that energizes baroreflex activation device 75 will be an electrical signal. The particular design of suitable electrodes are described in the referenced patents and applications, the full disclosures of which are hereby incorporated in by reference. One suitable form of baroreflex activation device includes an electrode assembly having at least one set of electrodes with a tripolar configuration. In an embodiment, the tripolar (or pseudo-tripolar) electrode set has two leads for applying a voltage across a baroreceptor and/or nerve fibers that carry signals from the baroreceptor to the brain. An embodiment of such a tripolar electrode is described in the above-referenced application Ser. No. 10/402,911 (Attorney Docket No. 21433-000410), the full disclosure of which is incorporated herein by reference in its entirety. However, it should be appreciated that the methods and devices of the present invention may be used with any number of electrodes and configurations, as for example a bipolar electrode, or a monopolar electrode (e.g., an electrode set including an active electrode and a dispersive electrode). For further details of exemplary electrodes useful in the practice of the present invention, reference may be made to U.S. patent application Ser. Nos. 10/402,911 (Attorney Docket No. 21433-000410US), filed Mar. 27, 2003 (e.g., FIG. 27); 10/402,393 (Attorney Docket No. 21433-000420US), filed Mar. 27, 2003; and 10/958,694 (Attorney Docket No. 21433-001600US), filed Oct. 4, 2004; the full disclosures of all of which were previously incorporated by reference in their entirety.

Baroreflex activation device 75 is suitable for implantation, and is preferably implanted using a minimally invasive percutaneous transluminal approach and/or a minimally invasive surgical approach. Baroreflex activation device 75 may be positioned anywhere that baroreceptors 40 affecting baroreflex system 50 are numerous, such as in the heart 12, in the aortic arch 15, in the common carotid arteries 20/22 near the carotid sinus 35, in the subclavian arteries 17/25, in the brachiocephalic artery 30, in the femoral and/or iliac arteries (not shown), in the veins (not shown), or in the cardiopulmonary region (not shown). Baroreflex activation device 75 may be implanted such that it is positioned adjacent baroreceptors 40 and/or nerve fibers that carry signals from the baroreceptor to the brain. Alternatively, baroreflex activation device 75 may be outside the body such that the device is positioned a short distance from but proximate to baroreceptors 40 and/or nerve fibers that carry signals from the baroreceptor to the brain. Preferably, baroreflex activation device 75 is implanted near the right carotid sinus 35 and/or the left carotid sinus (near the bifurcation of the common carotid artery) and/or the aortic arch 15, where baroreceptors 40 and/or nerve fibers that carry signals from the baroreceptor to the brain have a significant impact on baroreflex system 50, or in the pulmonary artery.

For purposes of illustration only, the present invention is described with reference to baroreflex activation device 75 positioned near the carotid sinus 35. Furthermore, for clarity, FIG. 3 shows a single baroreflex activation device 75. However, it is believed that advantages can be achieved by providing two or more baroreflex activation devices, and energizing them in a synchronous, sequential, or alternating manner. For example, similar devices could be positioned in both carotid sinus regions (or other regions), and driven alternately. This will be described in greater detail below.

Baroreflex Receptor Stimulus Methods

In an embodiment, a method for stimulating the baroreceptors 40 includes establishing a suitable therapy regimen which delivers at least one pulse, preferably more than one, to one or more of the baroreceptors. In an embodiment, a plurality of pulses are delivered. At least one of the one or more pulses includes two or more distinct phases, with each phase having a polarity which is different than the other phase of the same pulse.

In an embodiment, each phase is delivered for a predefined duration of time (i.e., predefined phase width). Each phase of the each pulse is separated by an interphase delay of predefined time period (i.e., interphase delay width). The phase width of each pulse may be similar or different from the phase width of the other phase of the same pulse. For example, the first phase of a given pulse may have an equal, shorter, or greater phase width (time duration) than the second phase of the same pulse. Similarly, the phase width may be similar, less, or more than the interphase delay between two successive phases of the same pulse. It should be appreciated that the various phases of the regimen therapy (both inter-pulse and intra-pulse) may have similar or different waveforms as for example different amplitudes, widths, size, and shapes (e.g., square wave or ramp wave, symmetrical or asymmetrical). For example, the phase widths and the interphase delays of different pulses may be similar or different from one another. In some embodiments, the interphase delay between two phases of a given pulse may be equal, shorter, or greater than the time duration between that pulse and another pulse immediately preceding or following that given pulse. In an embodiment, the therapy regimen includes a series of biphasic pulses with the interphase delay between two successively delivered phases being shorter than a time interval between the two adjacently delivered (i.e., two pulses delivered immediately next to each other) biphasic pulses. Alternatively, the interphase delay between two successively delivered phases may be greater than or equal to the time interval between the two adjacently delivered biphasic pulses. In an embodiment, the width of the first phase is effectively shorter than that of the second phase to equilibrate the charge delivered to the baroreflex system in each phase.

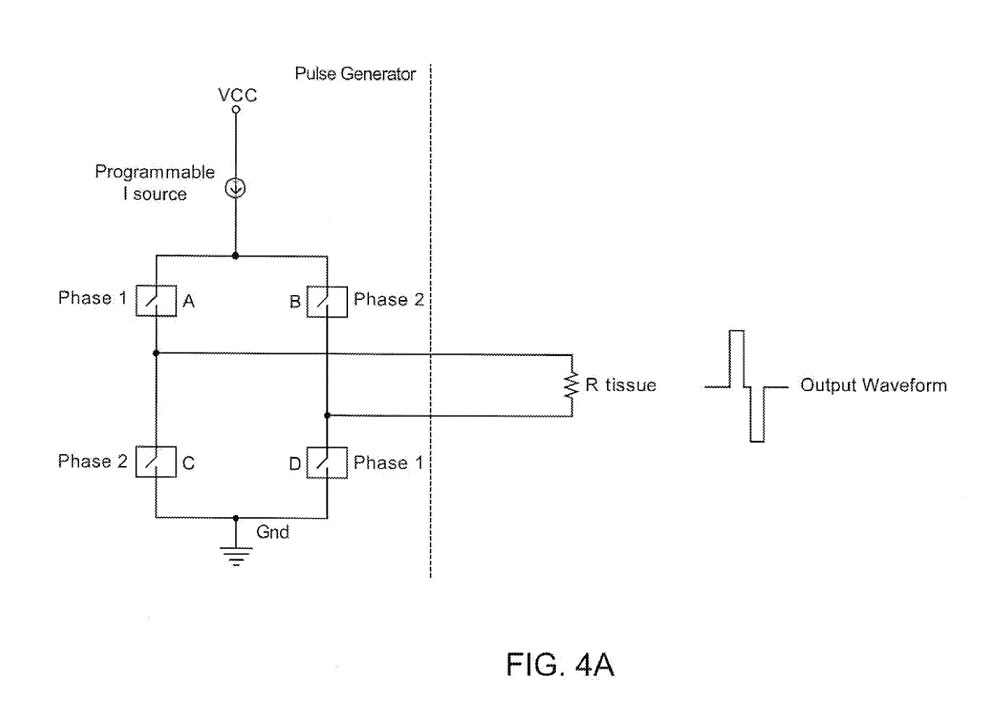

Now referring to FIG. 4A, an exemplary circuit diagram, and the corresponding output waveform generated as a result of the operation of that circuit, are shown for a single pulse of a biphasic output, usable in the practice of the invention. It should, of course, be appreciated by those skilled in the art, that one or more phases may be used during the practice of the invention. In the embodiment features of which are shown in FIG. 4, during a first phase (Ph1) of a pulse (Pu1), switches A and D are closed (thus able to allow passage of current) and switches B and C are open (thus preventing passage of current). Current flows through switch A to and enters a load or tissue (R) in one direction, and travels and flows through switch D. This phase (Ph1) produces the positive first phase of the pulse.

During a second phase (Ph2) of the pulse 1 (Pu1), switches B and C are closed (thus able to allow passage of current) and switches A and D are open (thus preventing passage of current). Current flows through switch B to and enters the load or tissue (R) in another direction opposite that of the one direction, and exits the load and continues to flow through switch C. This phase (Ph2) produces the negative second phase of the pulse. Thus, for at least one pulse during the therapy regimen, the polarity of the at least one electrode switches from behaving as a cathode to an anode and/or vice versa.

In an embodiment, the magnitude of the each phase width and/or the interphase delay, independently, may range from about 30 to about 3000 micro seconds (“μs”), from about 100 to about 1000 μs, from about 200 to about 2000 μs, from about 500 to about 3000 μs, from about 30 to about 500 μs. In an embodiment, the phase width and/or the interphase delay, is about 100 μs. In an embodiment, the biphasic pulse comprises the output of a single discharging capacitor and the polarity is switched midway during the delivery of the pulse such that the first phase and the second phase have equal widths. In an embodiment, the interphase delay is about 100 μs.

The biphasic method according to the present invention and as shown in FIG. 4A, may be used with a constant voltage output by replacing the constant current source (as shown in FIG. 4A) with a voltage source. An exponential decaying voltage pulse could also be used by replacing the current source with a charged capacitor.

It should be appreciated that the various phases of the regimen therapy (both inter-pulse and intra-pulse) may have similar or different waveforms as for example different amplitudes, widths, size, and shapes (e.g., square wave or ramp wave, symmetrical or asymmetrical); some of which are shown in FIGS. 4B-4F.

It should be appreciated that the methods and devices of the present invention may be used with any number of electrodes and configurations, as further described below.

Representative Sensors

While sensor 80 is optional, and embodiments of the invention can operate without using such a sensor, the sensor is a useful feature, and several representative types will be discussed. Sensor 80 may comprise any suitable device that measures or monitors a parameter indicative of the need to modify the activity of the baroreflex system. For example, sensor 80 may comprise a physiologic transducer or gauge that measures ECG, blood pressure (systolic, diastolic, average or pulse pressure), blood volumetric flow rate, blood flow velocity, respiration, blood pH, oxygen or carbon dioxide content, mixed venous oxygen saturation (SVO₂), vasoactivity, nerve activity, tissue activity, or tissue or blood composition. Examples of suitable transducers or gauges for sensor 80 include ECG electrodes, a piezoelectric pressure transducer, an ultrasonic flow velocity transducer, an ultrasonic volumetric flow rate transducer, a thermodilution flow velocity transducer, a capacitive pressure transducer, an impedance sensor, a membrane pH electrode, an optical detector (SVO₂) or a strain gauge. Although only one sensor 80 is shown, multiple sensors 80 of the same or different type at the same or different locations may be utilized.

An example of an implantable blood pressure measurement device that may be disposed about a blood vessel is disclosed in U.S. Pat. No. 6,106,477 to Miesel et al. An example of a subcutaneous ECG monitor is available from Medtronic under the trade name REVEAL ILR and is disclosed in PCT Publication No. WO 98/02209. Other examples are disclosed in U.S. Pat. Nos. 5,987,352 and 5,331,966. Examples of devices and methods for measuring absolute blood pressure utilizing an ambient pressure reference are disclosed in U.S. Pat. No. 5,810,735 to Halperin et al., U.S. Pat. No. 5,904,708 to Goedeke, and PCT Publication No. WO 00/16686 to Brockway et al. Sensor 80 described herein may take the form of any of these devices or other devices that generally serve the same purpose. The full disclosures of all of the above were previously incorporated by reference in their entirety.

Sensor 80 is preferably positioned in a chamber of the heart 12, or in/on a major artery such as the aortic arch 15, a common carotid artery 20/22, a subclavian artery 17/25 or the brachiocephalic artery 30, such that the parameter of interest may be readily ascertained. Sensor 80 may be disposed inside the body such as in or on an artery, a vein or a nerve (e.g., vagus nerve), or disposed outside the body, depending on the type of transducer or gauge utilized. Sensor 80 may be separate from baroreflex activation device 75 or combined therewith. For purposes of illustration only, sensor 80 is shown positioned on the right subclavian artery 17.

Control System

Memory 87 may contain data related to the sensor signal, the control signal, and/or values and commands provided by input device 95. Memory 87 may also include software containing one or more algorithms defining one or more functions or relationships between the control signal and the sensor signal. The algorithm may dictate activation or deactivation control signals depending on the sensor signal or a mathematical derivative thereof. The algorithm may dictate an activation or deactivation control signal when the sensor signal falls below a lower predetermined threshold value, rises above an upper predetermined threshold value or when the sensor signal indicates a specific physiologic event. The algorithm may dynamically alter the threshold value as determined by the sensor input values.

Control system 72 may operate as a closed loop utilizing feedback from sensor 80, or other sensors, such as heart rate sensors which may be incorporated on the electrode assembly, or as an open loop utilizing reprogramming commands received by input device 95. The closed loop operation of control system 72 preferably utilizes some feedback from sensor 80, but may also operate in an open loop mode without feedback. Programming commands received by input device 95 may directly influence the control signal, the output activation parameters, or may alter the software and related algorithms contained in memory 87. The treating physician and/or patient may provide commands to input device 95. Display 97 may be used to view the sensor signal, control signal and/or the software/data contained in memory 87.

The control signal generated by control system 72 may be continuous, periodic, alternating, episodic, or a combination thereof, as dictated by an algorithm contained in memory 87. Continuous control signals include a constant pulse, a constant train of pulses, a triggered pulse and a triggered train of pulses. Examples of periodic control signals include each of the continuous control signals described above which have a designated start time (e.g., beginning of each period as designated by minutes, hours, or days in combinations of) and a designated duration (e.g., seconds, minutes, hours, or days in combinations of). Examples of alternating control signals include each of the continuous control signals as described above which alternate between the right and left output channels. Examples of episodic control signals include each of the continuous control signals described above which are triggered by an episode (e.g., activation by the physician/patient, an increase/decrease in blood pressure above a certain threshold, heart rate above/below certain levels, respiration, etc.).

Exemplary Electrode Assembly

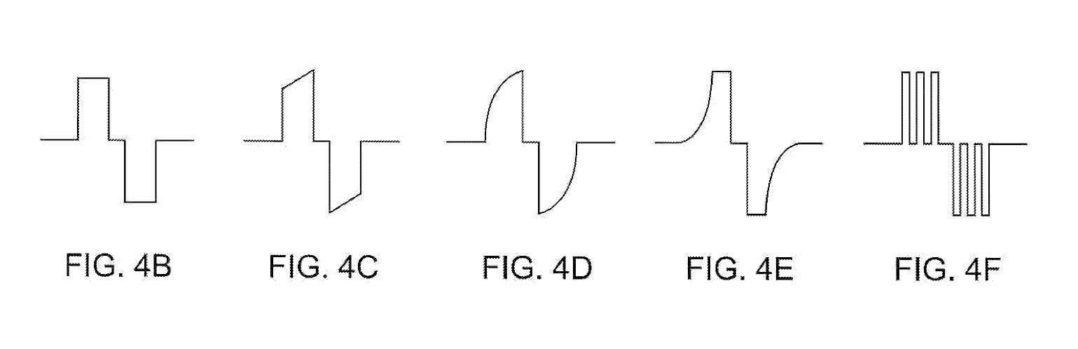

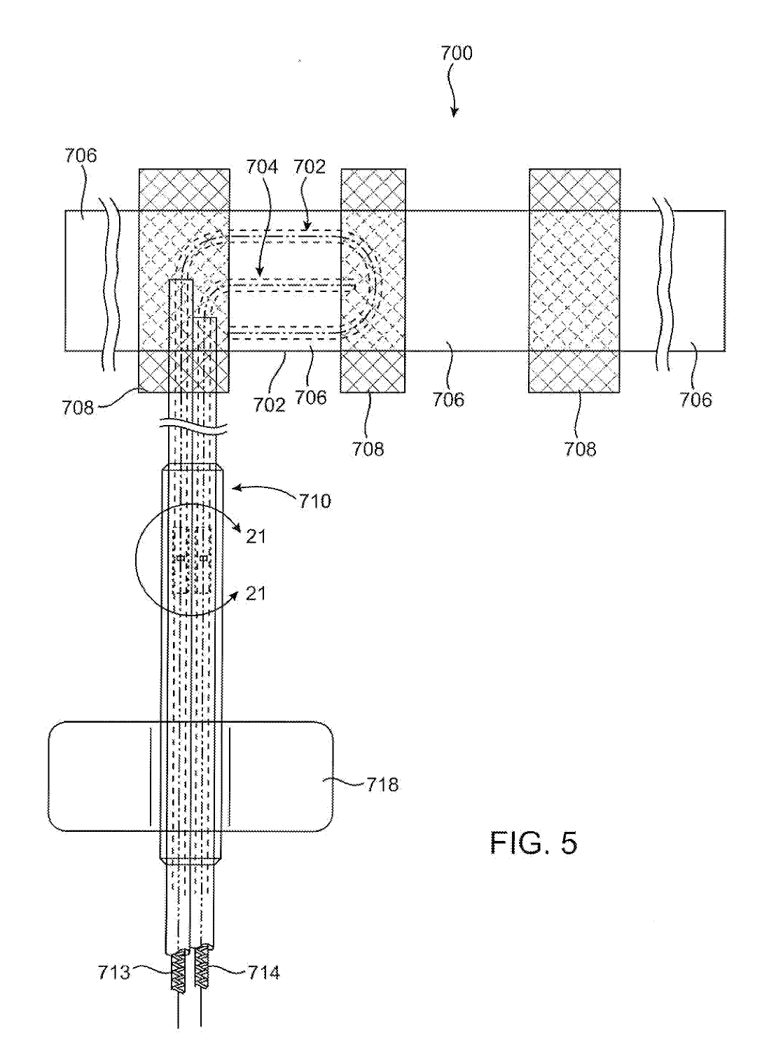

Now referring to FIGS. 5 and 6, an exemplary electrode assembly or cuff device 700, embodying features of the invention is shown, and generally includes coiled electrode conductors 702/704 embedded in a flexible support 706. In the embodiment shown, an outer electrode coil 702 and an inner electrode coil 704 are used to provide a pseudo-tripolar arrangement (e.g., two leads wherein two of the three electrodes are electronically coupled), but other polar arrangements are applicable as well as those described in previously-referenced patents and/or patent applications. The coiled electrodes 702/704 may be formed of fine round, flat or ellipsoidal wire such as 0.002 inch diameter round PtIr alloy wire wound into a coil form having a nominal diameter of 0.015 inches with a pitch of 0.004 inches, for example. The flexible support or base 706 may be formed of a biocompatible and flexible (preferably elastic) material such as silicone or other suitable thin walled elastomeric material having a wall thickness of 0.005 inches and a length (e.g., 2.95 inches) sufficient to surround the carotid sinus, for example.

Each turn of the coil in the contact area of the electrodes 702/704 is exposed from the flexible support 706 and any adhesive to form a conductive path to the artery wall. The exposed electrodes 702/704 may have a length (e.g., 0.236 inches) sufficient to extend around at least a portion of the carotid sinus, for example. The long axis of the exposed electrode conductors may be parallel or perpendicular to the long axis of the vessel around or in which they are placed. The electrode cuff 700 is assembled flat with the contact surfaces of the coil electrodes 702/704 tangent to the inside plane of the flexible support 706. When the electrode cuff 700 is wrapped around the artery, the inside contact surfaces of the coiled electrodes 702/704 are naturally forced to extend slightly above the adjacent surface of the flexible support, thereby improving contact to the artery wall.

The ratio of the diameter of the coiled electrodes 702/704 to the wire diameter is preferably large enough to allow the coil to bend and elongate without significant bending stress or torsional stress in the wire. Flexibility is a significant advantage of this design which allows the electrode cuff 700 to conform to the shape of the carotid artery and sinus, and permits expansion and contraction of the artery or sinus without encountering significant stress or fatigue. In particular, the flexible electrode cuff 700 may be wrapped around and stretched to conform to the shape of the carotid sinus and artery during implantation. This may be achieved without collapsing or distorting the shape of the artery and carotid sinus due to the compliance of the electrode cuff 700. The flexible support 706 is able to flex and stretch with the conductor coils 702/704 because of the absence of fabric reinforcement in the electrode contact portion of the cuff 700. By conforming to the artery shape, and by the edge of the flexible support 706 sealing against the artery wall, the amount of stray electrical field and extraneous stimulation will likely be reduced.

The pitch of the coil electrodes 702/704 may be greater than the wire diameter in order to provide a space between each turn of the wire to thereby permit bending without necessarily requiring axial elongation thereof. For example, the pitch of the contact coils 702/704 may be 0.004 inches per turn with a 0.002 inch diameter wire, which allows for a 0.002 inch space between the wires in each turn. The inside of the coil may be filled with a flexible adhesive material such as silicone adhesive which may fill the spaces between adjacent wire turns. By filling the small spaces between the adjacent coil turns, the chance of pinching tissue between coil turns is minimized thereby avoiding abrasion to the artery wall. Thus, the embedded coil electrodes 702/704 are mechanically captured and chemically bonded into the flexible support 706. In the unlikely event that a coil electrode 702/704 comes loose from the support 706, the diameter of the coil is large enough to be atraumatic to the artery wall. Preferably, the centerline of the coil electrodes 702/704 lie near the neutral axis of electrode cuff structure 700 and the flexible support 706 comprises a material with isotropic elasticity such as silicone in order to minimize the shear forces on the adhesive bonds between the coil electrodes 702/704 and the support 706.

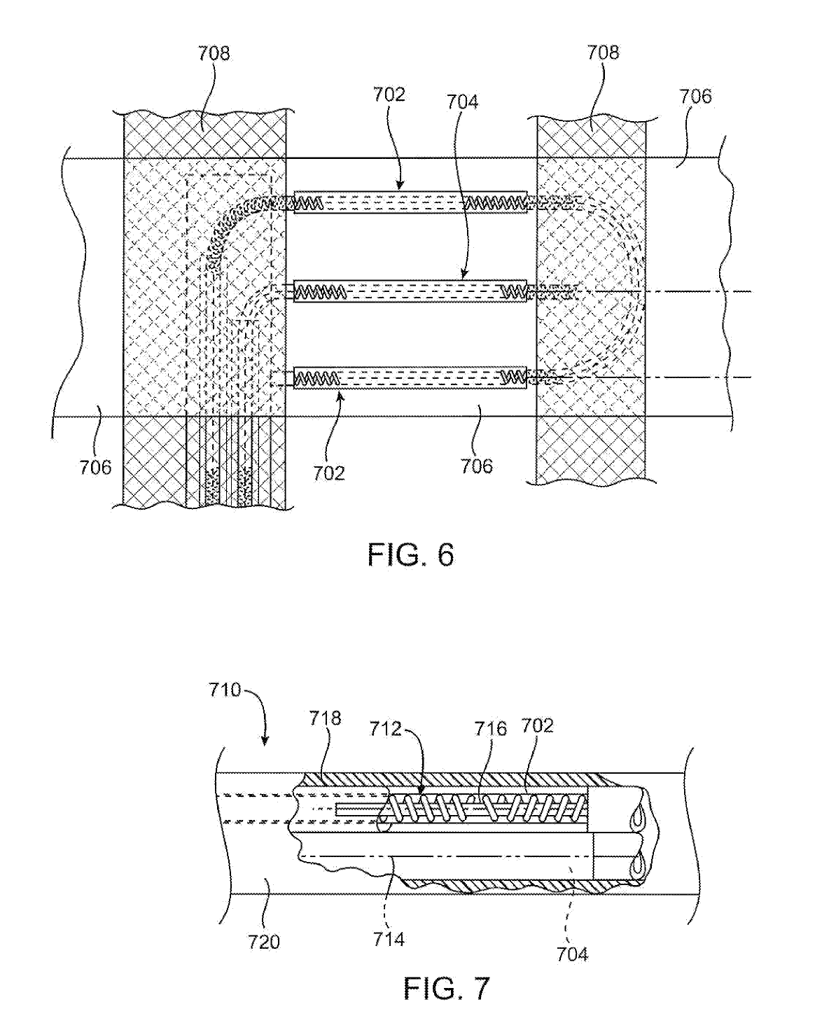

The electrode coils 702/704 are connected to corresponding conductive coils 712/714, respectively, in an elongate lead 710 which is connected to the control system 60. Anchoring wings 718 may be provided on the lead 710 to tether the lead 710 to adjacent tissue and minimize the effects or relative movement between the lead 710 and the electrode cuff 700. As seen in FIG. 7, the conductive coils 712/714 may be formed of 0.003 MP35N bifilar wires wound into 0.018 inch diameter coils which are electrically connected to electrode coils 702/704 by splice wires 716. The conductive coils 712/714 may be individually covered by an insulating covering 718 such as silicone tubing and collectively covered by insulating covering 720.

However, it should be appreciated that the methods and devices of the present invention may be used with any number of electrodes and configurations, as for example a bipolar electrode, or a monopolar electrode (e.g., an electrode set including an active electrode and a dispersive electrode), tripolar, and pseudo-tripolar, and combinations thereof. For further details of exemplary electrodes useful in the practice of the present invention, reference may be made to U.S. patent application Ser. Nos. 10/402,911 (Attorney Docket No. 21433-000410US), filed Mar. 27, 2003; 10/402,393 (Attorney Docket No. 21433-000420US), filed Mar. 27, 2003; and 10/958,694 (Attorney Docket No. 21433-001600US), filed Oct. 4, 2004; the full disclosures of all of which were previously incorporated by reference in their entirety. By way of example, FIG. 27 of U.S. patent application Ser. No. 10/402,393 (Attorney Docket No. 21433-000420US) illustrates another suitable electrode.

For further details of exemplary baroreflex activation devices, reference may be made to U.S. Pat. Nos. 6,522,926, 6,616,624, 6,985,774, 7,158,832, 6,850,801; and U.S. patent application Ser. Nos. 10/284,063, 10/453,678, 10/402,911, 10/402,393, 10/818,738, and 60/584,730, 10/958,694; the full disclosures of all of which were previously incorporated by reference in their entirety.

Although the above description provides a complete and accurate representation of the invention, the present invention may be manifested in a variety of forms other than the specific embodiments described and contemplated herein. Accordingly, departures in form and detail may be made without departing from the scope and spirit of the present invention as described in the appended claims.

What is claimed is:

1. A method for stimulation of a baroreflex system of a patient, comprising:
establishing a therapy regimen comprising at least one pulse including at least two phases wherein each phase has a polarity which is different than that of another phase within the same pulse; and
activating a baroreflex system of the patient with at least one baroreflex activation device responsive to the therapy regimen.

2. A method according to claim 1, wherein the therapy regimen includes a plurality of pulses.

3. A method according to claim 2, wherein the plurality of the pulses comprises at least one biphasic pulse.

4. A method according to claim 2, wherein the phases of at least one given pulse change polarity at least once during the given pulse.

5. A method according to claim 2, wherein each phase has a predefined phase width.

6. A method according to claim 5, wherein each phase is separated by an interphase delay.

7. A method according to claim 5, wherein the first phase and the second phase have equal phase widths.

8. A method according to claim 7, wherein the interphase delay is about 100 micro seconds (“μs”).

9. A method according to claim 5, wherein the first phase has a shorter phase width than the second phase.

10. A method according to claim 5, wherein the first phase has a longer phase width than the second phase.

11. A method according to claim 5, wherein the width of the first phase is effectively shorter than that of the second phase to equilibrate the charge delivered to the baroreflex system in each phase.

12. A method according to claim 6, wherein the therapy regimen comprises a series of biphasic pulses each comprising two phases of stimulation delivered in succession with an interphase delay that is shorter than a time interval between two successive biphasic pulses.

13. A method according to claim 6, wherein each phase width and/or the interphase delay, independently, ranges from about 30 to about 3000 micro seconds (“μs”).

14. A method according to claim 6, wherein each phase width and/or the interphase delay, independently, ranges from about 100 to about 1000 μs.

15. A method according to claim 6, wherein each phase width and/or the interphase delay, independently, ranges from about 200 to about 2000 μs.

16. A method according to claim 6, wherein each phase width and/or the interphase delay, independently, ranges from about 500 to about 3000 μs.

17. A method according to claim 6, wherein each phase width and/or the interphase delay, independently, ranges from about 30 to about 500 μs.

18. A method according to claim 6, wherein each pulse width and the interphase delay is about 100 μs.

19. A method according to claim 2, wherein during one phase of a given pulse, current flows through the target baroreflex system in one direction and produces a positive phase.

20. A method according to claim 19, wherein during another phase of the given pulse, current flows through the target baroreflex system in another direction opposite the one direction and produces a negative phase.

21. A method according to claim 19, wherein the positive phase is the first phase of the biphasic pulse.

22. A method according to claim 19, wherein the positive phase is the second phase of the biphasic pulse.

23. A method according to claim 3, wherein each phase of the plurality of the biphasic pulses always starts with the same polarity.

24. A method according to claim 3, wherein the polarity of the first phase of the plurality of the biphasic pulses alternates between positive and negative.

25. A method for stimulation of a baroreflex system of a patient, comprising:
providing an electrode assembly comprising at least one electrode configured to behave, alternatively, as a cathode and an anode;
applying a therapy regimen comprising at least one biphasic pulse to the electrode assembly with each phase of the biphasic pulse having a polarity which is different than the other phase of the same biphasic pulse; and
activating a baroreflex system of a patient with at least one baroreflex activation device responsive to the therapy regimen.

26. A method according to claim 25, wherein the electrode assembly comprises a plurality of electrodes.

27. A method according to claim 26, wherein the electrode assembly comprises at least one set of electrodes having a tripolar or pseudotripolar configuration.

28. A method according to claim 27, wherein the at least one tripolar or pseudotripolar electrode set includes a central electrode and two outer electrodes.

29. A method according to claim 28, wherein at nominal polarity, the central electrode has a different charge than the outer two electrodes.

30. A method according to claim 29, wherein at nominal polarity the central electrode behaves as a cathode and the outer two electrodes behave as anodes.

31. A method according to claim 26, wherein the polarity of each electrode is changed at least once during the at least one pulse.

32. A method according to claim 26, wherein the therapy regimen comprises a plurality of pulses.

33. A method according to claim 32, wherein each electrode starts a next pulse with a polarity which is the same as that at a previous pulse.

34. A method according to claim 32, wherein each electrode starts a next pulse with a polarity which is different than that at a previous pulse.

35. A device for activating a baroreflex system of a patient, comprising:
an electrode assembly comprising at least one electrode configured to be responsive to a therapy regimen which applies at least one pulse including at least two phases wherein each phase of the at least one pulse imparts to a given electrode a polarity which is different from that imparted to the same given electrode by another phase of the same pulse.

36. A device according to claim 35, wherein the therapy regimen comprises a plurality of pulses.

37. A device according to claim 36, wherein the plurality of the pulses comprises at least one biphasic pulse.

38. A device according to claim 37, wherein the at least one electrode changes polarity at least once during the given pulse.

39. A device according to claim 37, wherein the electrode assembly comprises at least one set of electrodes having a tripolar or pseudotripolar configuration.

40. A device according to claim 39, wherein the at least one tripolar or pseudotripolar electrode set includes a central electrode and two outer electrodes.

41. A device according to claim 40, wherein at nominal polarity, the central electrode has a different charge than the outer two electrodes.

42. A device according to claim 40, wherein at nominal polarity the central electrode behaves as a cathode and the outer two electrodes behave as anodes.

43. A device according to claim 39, wherein the polarity of each electrode is at least once changed during the therapy regimen.

44. A device according to claim 35, wherein a source of energy for the device is a voltage source, current source, or charged capacitor.

45. A device according to claim 44, wherein the current source is a constant current source.
